(19) United States
(12) Patent Application Publication
(10) Pub. No.: US 2015/0182447 A1
(43) Pub. Date: Jul. 2, 2015

(54) METHOD FOR IMPROVING SKIN CONDITION COMPRISING SIX STEPS AND SYSTEM THEREFOR

(71) Applicant: Soung Lyul Park, Daejeon (KR)

(72) Inventor: Soung Lyul Park, Daejeon (KR)

(21) Appl. No.: 14/657,468

(22) Filed: Mar. 13, 2015

(30) Foreign Application Priority Data
Oct. 4, 2013 (KR) 10-2013-0118345

(51) Int. Cl. A61K 8/97; A61K 8/49; A61K 8/64; A61K 8/368; A61Q 19/02; A61K 8/41; A61K 8/58; A61Q 19/10; A61Q 19/08; A61K 8/73; A61K 8/34

(52) U.S. Cl. CPC A61K 8/97; A61K 8/73; A61K 8/4953; A61K 8/735; A61K 8/64; A61K 8/368; A61K 8/345; A61K 8/416; A61K 8/585; A61K 8/4973; A61Q 19/10; A61Q 19/08; A61Q 19/02; A61K 2800/884

(57) ABSTRACT

The present disclosure relates to a method and a system for skin care using a soap, a lifting pack, an eye cream, a whitening essence, a nourishing cream and a blemish balm or beauty balm cream. By using the six products in sequence, their individual effects can be maximized, thereby providing healthy, bright and transparent skin and restoring skin aging.

More specifically, the method and the system according to the present disclosure includes a soap for effective face cleansing, a pack for lifting skin, an eye cream for improving wrinkles, an essence for whitening skin, a nourishing cream for providing skin regeneration ability and a blemish balm or beauty balm cream for protecting skin.

This utility application claims priority to Korean Patent No. 10-2013-0118345, filed Oct. 4, 2013, and is incorporated herein by reference.

### BACKGROUND OF THE INVENTION

1. Technical Field

The present disclosure relates to a facial cleanser, a pack, an eye cream, an essence, a nourishing cream and a blemish balm or beauty balm cream for improving skin-related troubles in a cosmetic aspect. More particularly, it relates to a method and a system for maximizing the effect of each product by using them in sequence.

2. Related Art

Wastes and contaminants are accumulated in skin every day. Also, the skin develops fine wrinkles and loses elasticity with aging. There have been many efforts for maintenance of skin hygiene and prevention of skin aging. They mostly relate to cosmetic compositions containing ingredients effective in hygiene and prevention of skin aging. However, skin wrinkles or skin elasticity cannot be improved effectively only by simply applying the cosmetic composition on skin. Moreover, although most women use several, not one, facial cleansers or other cosmetic products a day, the effect each of the product has on one another has not been considered.

### SUMMARY OF THE INVENTION

(Patent document 1) Korean Patent Registration No. 10-0897364, which relates to a functional natural cosmetic soap and a functional mask pack, describes a cosmetic composition not containing a chemical ingredient generally used in soap such as a surfactant. However, it merely describes a cosmetic composition that can be used for a soap or a mask pack.

(Patent document 2) Korean Patent Publication No. 10-2008-0083596 describes a cosmetic mask pack effective for preventing skin aging and treating dermatitis such as atopy, acne, etc. However, the mask pack is limited to the problem of excessive sebum removal caused by frequent use of soap.

(Patent document 3) Korean Patent Publication No. 10-2005-0034005 describes a nourishing cream for massage and a massage pack using the same. However, it does not relate to a facial cleanser, an eye cream or a blemish balm or beauty balm cream that can be used together with the nourishing cream or the massage pack.

(Patent document 4) Korean Patent Registration No. 10-1210371 describes a blemish balm or beauty balm cream and a suncare makeup cosmetic composition containing a stabilized emulsion of an inorganic UV protection agent. However these are not formulated to be used together with other cosmetics.

(Patent document 5) Korean Patent Publication No. 10-2006-0103790 describes a cosmetic kit and a skin massage method for preventing skin aging. However, it is restricted to a kit containing a cosmetic composition and a massage method using the same.

The present disclosure is directed to providing a method and a system for skin improvement and care using a facial cleanser, a lifting pack, an eye cream, an essence, a nourishing cream and a blemish balm or beauty balm cream in sequence to maximize and optimize the intrinsic effect of each cosmetic, i.e., skin cleansing, skin lifting, wrinkle improvement, skin whitening, skin regeneration and wound healing, and to provide a synergic effect.

The present disclosure provides a method for improving skin, including, in sequence, cleansing the face with a soap containing herbal ingredients, following the face cleansing, applying a lifting pack containing purified water, a carbomer, oriental raisin tree extract, yeast β-glucan, caffeine, oriental raisin fruit extract, green tea extract, lonicera flower extract, peony extract, sophora root extract, scutellaria root, nutgall extract, peach leaf extract, ginko leaf extract, coconut oil, tea tree oil, aloe, arginine and hyaluronic acid and further containing one or more selected from a group consisting of acetyl hexapeptide-8, copper tripeptide-1, p-anisic acid and caprylyl glycol to the skin, following the application of the lifting pack, applying an eye cream containing purified water, a glyceryl acrylate/acrylic acid copolymer and glycerin and further containing one or more selected from a group consisting of propylene glycol, butylene glycol, betaine, PEG-8 dimethicone, Polysorbate 20, PPG-26-Buteth-26, cyclopentasiloxane, cyclotetrasiloxane, dimethiconol and potassium glycyrrhizate around the eyes, following the application of the eye cream, applying a whitening essence containing purified water, dipropylene glycol, squalane, glycerin and arbutin and further containing one or more selected from a group consisting of phenyl trimethicone, a PEG-10 dimethicone/vinyl dimethicone crosspolymer, hydrogenated lecithin, cyclopentasiloxane, 1,2-hexanediol, polyglyceryl-3 methylglucose distearate, sodium hydrogenated tallow glutamate, a carbomer, ethoxydiglycol, xanthan gum and a trimethylpentanediol/adipic acid copolymer, following the application of the essence, applying a nourishing cream containing purified water, dipropylene glycol, cetyl ethylhexanoate, a PEG-10 dimethicone/vinyl dimethicone crosspolymer and squalane and further containing one or more selected from a group consisting of glyceryl stearate, dimethicone, 1,2-hexanediol, stearyl alcohol, meadowfoam seed oil, Polysorbate 60, ethoxydiglycol, a trimethylpentanediol/adipic acid copolymer, hydrogenated lecithin, sodium hydrogenated tallow glutamate, a carbomer and cetyl alcohol, and, following the application of the nourishing cream, applying a blemish balm or beauty balm cream containing purified water, caprylic/capric triglyceride, titanium dioxide, propylene glycol and squalane and further containing one or more selected from a group consisting of cetyl PEG/PPG-10/1-dimethicone, arbutin, butylene glycol dicaprylate/dicaprate, sorbitan sesquioleate, magnesium sulfate, calcium stearate and microcrystalline wax and a system for improving skin including the soap, the lifting pack, the eye cream, the whitening essence, the nourishing cream and the blemish balm or beauty balm cream used according to the method.

The present disclosure also provides a method and a system for improving skin, using a second formulation containing Na₂O, SiO₂ and liquid sodium silicate as a formulation mixed with the lifting pack.

More specifically, the present disclosure provides a method and a system for improving skin using an eye cream which further contains, in addition to the above-described ingredients, one or more selected from a group consisting of oriental raisin fruit extract, sodium hyaluronate, yeast β-glucan, acetyl hexapeptide-8, copper tripeptide-1, palmitoyl pentapeptide-4, PEG-40 hydrogenated castor oil, human oligopeptide-1, tripeptide-29, triethanolamine, adenosine, phenoxyethanol, 1,2-hexanediol benzophenone-5, diazolinyl urea and a fragrance.

The present disclosure also provides a method and a system for improving skin using a whitening essence which further contains, in addition to the above-described ingredients, one or more selected from a group consisting of tocopheryl acetate, oriental raisin fruit extract, sodium hyaluronate, sodium polyacrylate, yeast β-glycan, acetyl hexapeptide-8, copper tripeptide-1, palmitoyl pentapeptide-4, diipeptide-2, human oligopeptide-1, phenoxyethanol, triethanolamine, tetrasodium EDTA, a fragrance and a pigment.

In addition, the present disclosure provides a method and a system for improving skin using a nourishing cream which further contains, in addition to the above-described ingredients, one or more selected from a group consisting of tocopheryl acetate, xanthan gum, sodium hyaluronate, triethanolamine, palmitoyl pentapeptide-4, acetyl hexapeptide-8, copper tripeptide-1, benzophenone-5, dipotassium glycyrrhizate, sodium polyacrylate, yeast β-glucan, C13-14 isoparaffin, laureth-7, polyacrylamide, human oligopeptide-1, adenosine, tetrasodium EDTA and a fragrance.

The present disclosure also provides a method and a system for improving skin using a blemish balm or beauty balm cream which further contains, in addition to the above-described ingredients, one or more selected from a group consisting of white gourd fruit extract, portulaca extract, aloe vera leaf juice, oriental raisin fruit extract, nylon-12, disteardimonium hectorite, triethoxycaprylylsilane, beeswax, poly(methyl methacrylate), tocopheryl acetate, cellulose gum, dimethicone, copper tripeptide-1, acetyl hexapeptide-8, palmitoyl pentapeptide-4, DMDM hydantoin, phenoxyethanol, red iron oxide, yellow iron oxide, black iron oxide, ultramarine, sodium lauroyl glutamate, aluminum hydroxide and a fragrance.

A method and a system for skin improvement and care according to the present disclosure provide an effect of maximizing the intrinsic effects of a facial cleanser, a lifting pack, an eye cream, an essence, a nourishing cream and a blemish balm or beauty balm cream, which have been used independently.

### BRIEF DESCRIPTION OF THE DRAWING

FIG. 1 shows change in skin condition before and after use of a method and a system for skin care according to the present disclosure.

### DETAILED DESCRIPTION

Hereinafter, exemplary embodiments of the present disclosure and effects obtained therefrom are described in detail.

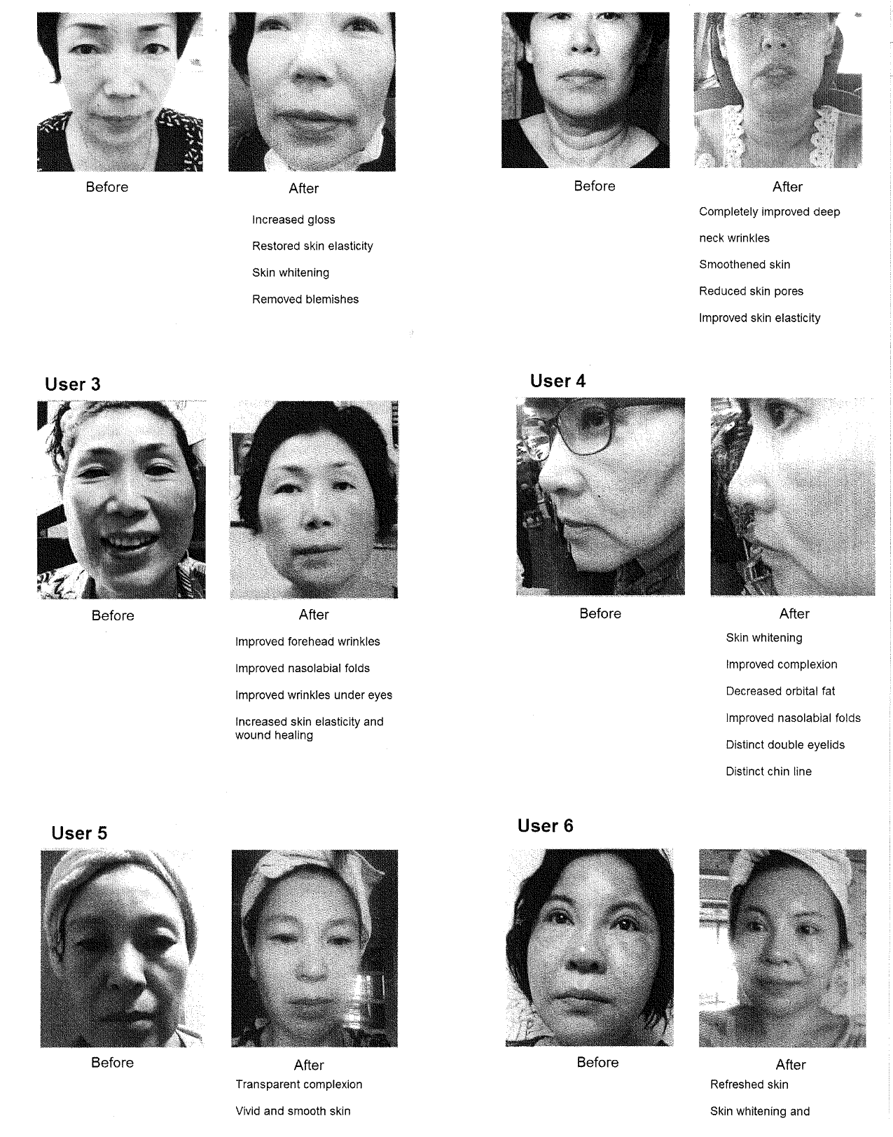

FIG. 1 shows change in the skin of users 1-6 who used a skin care method according to the present disclosure for at least a month. Specifically, the users 1-3 used a skin care system according to the present disclosure for a month and the users 4-6 used the skin care system according to the present disclosure for two months.

The user 1 showed increased gloss, restored skin elasticity, skin whitening and removed blemishes. The user 2 showed completely improved deep neck wrinkles as well as improved skin elasticity with smoothened skin and reduced skin pores. The user 3 showed improved forehead wrinkles, nasolabial folds and wrinkles under the eyes as well as increased skin elasticity and wound healing. The user 4 showed skin whitening, decreased orbital fat and decreased nasolabial folds. The user 5 showed more transparent complexion, improved skin texture and improved wrinkles under the eyes and nasolabial folds. The user 6 showed skin regeneration, improved blood circulation and skin tone and increased skin elasticity.

Each ingredient or step of a skin care system or method according to the present disclosure providing such effects is described in detail.

A facial cleanser used in the present disclosure may be a commonly used soap. By containing natural plant-derived seed oils and herbal ingredients, it produces soft foams during face cleansing, keeps the skin moist even after the foams are washed off with water and effectively removes wastes and leftovers, thereby smoothening the skin as in the user 2 shown in FIG. 1. More specifically, the facial cleanser of the present disclosure may contain at least 90% of a soap base consisting of about 75% of seed oil and about 25% of pam oil and one or more of 22 herbal ingredients, chamomile, alkoxyglycerol, hyaluronic acid, aloe vera extract, arginine, grape seed oil, tea tree oil, a skin moisturizer and a fragrance as the remainder. More specifically, it may contain all of the above-described ingredients.

A lifting pack used in the present disclosure is applied to the skin, particularly to the face, in the form of cream, emulsion or liquid, unlike the existing patch-type formulation. With time, it is dried and hardened as it contacts with air. By relaxing capillary vessels and degrading subcutaneous fat, it allows the skin to breathe and more easily accept nutrients provided from the products or steps that will be described later, thereby restoring skin elasticity as in the users 1-3 shown in FIG. 1. The effect of the lifting pack can be maximized when it is used after face cleansing with the soap according to the present disclosure. The lifting pack according to the present disclosure contains purified water, a carbomer, oriental raisin tree extract, yeast β-glucan, caffeine, oriental raisin fruit extract, green tea extract, lonicera flower extract, peony extract, sophora root extract, scutellaria root, nutgall extract, peach leaf extract, ginko leaf extract and hyaluronic acid as main ingredients and may further contain, as known ingredients for a pack, one or more selected from a group consisting of acetyl hexapeptide-8, copper tripeptide-1, p-anisic acid and caprylyl glycol. More specifically, it may contain all of the above-described ingredients.

Specifically, the lifting pack according to the present disclosure may be adjusted to pH 3.5±0.5.

The lifting pack according to the present disclosure may be applied to the skin after being mixed with a second formulation containing Na₂O, SiO₂ and liquid sodium silicate. When it is applied to the skin after being mixed with the second formulation, it can be more stably dried. Specifically, it is mixed with the second formulation before application to the skin, more specifically immediately before application to the skin.

The oriental raisin tree extract as the main ingredient of the lifting pack, which is known to be effective in relieving hangover, also moistens skin and provides uniform skin tone when used as a cosmetic ingredient. It is because the saponins present in abundance in the oriental raisin tree form skin-moisturizing films by binding to the sugar components of the skin horny layer. The oriental raisin tree extract may be obtained by any of extraction for a long time at high temperature, extraction at moderate temperature using ultrasonic waves or extraction for a short time under pressure.

An eye cream used in the present disclosure is applied around the eyes. Most specifically, it may be used after the lifting pack has been applied to the skin in order to supplement and supply enough nutrients to the skin. As a result, the effects of improving wrinkles around the eyes and degrading orbital fat can be maximized as in the users 3, 4 and 5 shown in FIG. 1.

The eye cream pack contains purified water, a glyceryl acrylate/acrylic acid copolymer and glycerin as main ingredients. It may further contain, as known ingredients of an eye cream, one or more selected from a group consisting of propylene glycol, butylene glycol, betaine, PEG-8 dimethicone, Polysorbate 20, PPG-26-Buteth-26, cyclopentasiloxane, cyclotetrasiloxane, dimethiconol and potassium glycyrrhizate. More specifically, it may contain all of the above-described ingredients.

More specifically, the eye cream may contain one or more selected from a group consisting of oriental raisin fruit extract, sodium hyaluronate, yeast β-glucan, acetyl hexapeptide-8, copper tripeptide-1, palmitoyl pentapeptide-4, PEG-40 hydrogenated castor oil, human oligopeptide-1, tripeptide-29, triethanolamine, adenosine, phenoxyethanol, 1,2-hexanediol benzophenone-5, diazolinyl urea and a fragrance. Most specifically, it may contain all of the above-described ingredients.

Specifically, the eye cream of the present disclosure may be adjusted to pH 5.0±0.5 and may have a specific gravity of 1.2±0.05 and a viscosity of 12,000±3,000.

An essence used in the present disclosure may be a whitening essence (or skin whitening essence) and may provide skin whitening effect while supplying or supplementing nutrients to the skin, particularly to the face, as can be seen in the users 1, 3, 4 and 6 shown in FIG. 1. For this purpose, the essence may be used after application of the eye cream, more specifically, after use of the soap, the lifting pack and the eye cream in sequence.

The essence contains purified water, dipropylene glycol, squalane, glycerin and arbutin and may further contain one or more selected from a group consisting of phenyl trimethicone, a PEG-10 dimethicone/vinyl dimethicone crosspolymer, hydrogenated lecithin, cyclopentasiloxane, 1,2-hexanediol, polyglyceryl-3 methylglucose distearate, sodium hydrogenated tallow glutamate, a carbomer, ethoxydiglycol, xanthan gum and a trimethylpentanediol/adipic acid copolymer. In addition to the above-described ingredients, it may further contain one or more selected from a group consisting of tocopheryl acetate, oriental raisin fruit extract, sodium hyaluronate, sodium polyacrylate, yeast β-glycan, acetyl hexapeptide-8, copper tripeptide-1, palmitoyl pentapeptide-4, diipeptide-2, human oligopeptide-1, phenoxyethanol, triethanolamine, tetrasodium EDTA, a fragrance and a pigment. Most specifically, it may contain all of the above-described ingredients.

Specifically, the whitening essence of the present disclosure may be adjusted to pH 6.0±0.5 and may have a specific gravity of 1.00±0.05 and a viscosity of 3,000±500.

A nourishing cream used in the present disclosure supplements and supplies sufficient nutrients to the skin and may provide natural skin regeneration ability and enhance skin immunity by containing EGF and a peptide at high concentrations, as can be seen in the users 1-6 shown in FIG. 1. The nourishing cream may be used after use of the whitening essence, more specifically, after use of the soap, the lifting pack, the eye cream and the whitening essence in sequence.

The nourishing cream contains purified water, EGF, a peptide, dipropylene glycol, cetyl ethylhexanoate, a PEG-10 dimethicone/vinyl dimethicone crosspolymer and squalane as main ingredients and may further contain one or more selected from a group consisting of glyceryl stearate, dimethicone, 1,2-hexanediol, stearyl alcohol, meadowfoam seed oil, Polysorbate 60, ethoxydiglycol, a trimethylpentanediol/adipic acid copolymer, hydrogenated lecithin, sodium hydrogenated tallow glutamate, a carbomer and cetyl alcohol. The nourishing cream may further contain one or more selected from a group consisting of tocopheryl acetate, xanthan gum, sodium hyaluronate, triethanolamine, palmitoyl pentapeptide-4, acetyl hexapeptide-8, copper tripeptide-1, benzophenone-5, dipotassium glycyrrhizate, sodium polyacrylate, yeast β-glucan, C13-14 isoparaffin, laureth-7, polyacrylamide, human oligopeptide-1, adenosine, tetrasodium EDTA and a fragrance. Most specifically, it may contain all of the above-described ingredients.

The nourishing cream of the present disclosure may be adjusted to pH 6.0±0.5 and may have a specific gravity of 1.10±0.05 and a viscosity of 20,000±5,000.

The EGF (epidermal growth factor) as one of the main ingredients of the nourishing cream is a growth factor that restores skin elasticity and promotes cell division. The nourishing cream of the present disclosure contains the EGF at high concentrations, i.e. 50 ppb or higher, specifically 70 ppb or higher, more specifically 100 ppb or higher. The EGF is involved in cell division which is basic for skin improvement and is known to provide the energy needed for cell division. The peptide as another main ingredient of the nourishing cream may contain 21 essential amino acids and may specifically contain adenosine, acetyl hexapeptide, palmitoyl peptide and copper tripeptide.

A blemish balm or beauty balm cream used in the present disclosure may be applied after application of the nourishing cream. Specifically, it may be used after application of the whitening essence and the nourishing cream in sequence, more specifically after use of the soap, the lifting pack, the eye cream, the whitening essence and the nourishing cream in sequence. In this case, it may provide natural-looking healthy and bright skin color and naturally cover skin defects. Also, since the blemish balm or beauty balm cream is an oil in water type formulation, it can effectively protect the skin from sweat or sebum.

The blemish balm or beauty balm cream contains purified water, caprylic/capric triglyceride, titanium dioxide, propylene glycol and squalane and may further contain one or more selected from a group consisting of cetyl PEG/PPG-10/1-dimethicone, arbutin, butylene glycol dicaprylate/dicaprate, sorbitan sesquioleate, magnesium sulfate, calcium stearate and microcrystalline wax. It may contain all of the above-described ingredients. More specifically, the blemish balm or beauty balm cream may further contain one or more selected from a group consisting of white gourd fruit extract, portulaca extract, aloe vera leaf juice, oriental raisin fruit extract, nylon-12, disteardimonium hectorite, triethoxycaprylylsilane, beeswax, poly(methyl methacrylate), tocopheryl acetate, cellulose gum, dimethicone, copper tripeptide-1, acetyl hexapeptide-8, palmitoyl pentapeptide-4, DMDM hydantoin, phenoxyethanol, red iron oxide, yellow iron oxide, black iron oxide, ultramarine, sodium lauroyl glutamate, aluminum hydroxide and a fragrance. Most specifically, it may contain all of the above-described ingredients.

The blemish balm or beauty balm cream according to the present disclosure may be adjusted to pH 6.0±1.0 and may have a specific gravity of 1.20±0.05 and a viscosity of 25,000±5,000.

What is claimed is:

1. A method for improving skin, comprising:
cleansing the face with a soap comprising herbal ingredients;
following the face cleansing, applying a lifting pack comprising purified water, a carbomer, oriental raisin tree extract, yeast β-glucan, caffeine, oriental raisin fruit extract, green tea extract, lonicera flower extract, peony extract, sophora root extract, scutellaria root, nutgall extract, peach leaf extract, ginko leaf extract and hyaluronic acid and further comprising one or more selected from a group consisting of acetyl hexapeptide-8, copper tripeptide-1, p-anisic acid and caprylyl glycol to the skin;
following the application of the lifting pack, applying an eye cream comprising purified water, a glyceryl acrylate/acrylic acid copolymer and glycerin and further comprising one or more selected from a group consisting of propylene glycol, butylene glycol, betaine, PEG-8 dimethicone, Polysorbate 20, PPG-26-Buteth-26, cyclopentasiloxane, cyclotetrasiloxane, dimethiconol and potassium glycyrrhizate around the eyes;
following the application of the eye cream, applying a whitening essence comprising purified water, dipropylene glycol, squalane, glycerin and arbutin and further comprising one or more selected from a group consisting of phenyl trimethicone, a PEG-10 dimethicone/vinyl dimethicone crosspolymer, hydrogenated lecithin, cyclopentasiloxane, 1,2-hexanediol, polyglyceryl-3 methylglucose distearate, sodium hydrogenated tallow glutamate, a carbomer, ethoxydiglycol, xanthan gum and a trimethylpentanediol/adipic acid copolymer;
following the application of the essence, applying a nourishing cream comprising purified water, EGF, a peptide, dipropylene glycol, cetyl ethylhexanoate, a PEG-10 dimethicone/vinyl dimethicone crosspolymer and squalane and further comprising one or more selected from a group consisting of glyceryl stearate, dimethicone, 1,2-hexanediol, stearyl alcohol, meadowfoam seed oil, Polysorbate 60, ethoxydiglycol, a trimethylpentanediol/adipic acid copolymer, hydrogenated lecithin, sodium hydrogenated tallow glutamate, a carbomer and cetyl alcohol; and
following the application of the nourishing cream, applying a blemish balm or beauty balm cream comprising purified water, caprylic/capric triglyceride, titanium dioxide, propylene glycol and squalane and further comprising one or more selected from a group consisting of cetyl PEG/PPG-10/1-dimethicone, arbutin, butylene glycol dicaprylate/dicaprate, sorbitan sesquioleate, magnesium sulfate, calcium stearate and microcrystalline wax.

2. The method according to claim 1, wherein the eye cream further comprises one or more selected from a group consisting of oriental raisin fruit extract, sodium hyaluronate, yeast β-glucan, acetyl hexapeptide-8, copper tripeptide-1, palmitoyl pentapeptide-4, PEG-40 hydrogenated castor oil, human oligopeptide-1, tripeptide-29, triethanolamine, adenosine, phenoxyethanol, 1,2-hexanediol benzophenone-5, diazolinyl urea and a fragrance.

3. The method according to claim 1, wherein the whitening essence further comprises one or more selected from a group consisting of tocopheryl acetate, oriental raisin fruit extract, sodium hyaluronate, sodium polyacrylate, yeast β-glycan, acetyl hexapeptide-8, copper tripeptide-1, palmitoyl pentapeptide-4, diipeptide-2, human oligopeptide-1, phenoxyethanol, triethanolamine, tetrasodium EDTA, a fragrance and a pigment.

4. The method according to claim 1, wherein the nourishing cream further comprises one or more selected from a group consisting of tocopheryl acetate, xanthan gum, sodium hyaluronate, triethanolamine, palmitoyl pentapeptide-4, acetyl hexapeptide-8, copper tripeptide-1, benzophenone-5, dipotassium glycyrrhizate, sodium polyacrylate, yeast β-glucan, C13-14 isoparaffin, laureth-7, polyacrylamide, human oligopeptide-1, adenosine, tetrasodium EDTA and a fragrance.

5. The method according to claim 1, wherein the blemish balm or beauty balm cream further comprises one or more selected from a group consisting of white gourd fruit extract, portulaca extract, aloe vera leaf juice, oriental raisin fruit extract, nylon-12, disteardimonium hectorite, triethoxycaprylylsilane, beeswax, poly(methyl methacrylate), tocopheryl acetate, cellulose gum, dimethicone, copper tripeptide-1, acetyl hexapeptide-8, palmitoyl pentapeptide-4, DMDM hydantoin, phenoxyethanol, red iron oxide, yellow iron oxide, black iron oxide, ultramarine, sodium lauroyl glutamate, aluminum hydroxide and a fragrance.

6. The method according to claim 1, wherein the lifting pack is applied to the skin after being mixed with a formulation comprising Na₂O, SiO₂ and liquid sodium silicate immediately before the application.

7. A system for improving skin, comprising:
a soap comprising herbal ingredients;
a lifting pack used after use of the soap, comprising purified water, a carbomer, oriental raisin tree extract, yeast β-glucan, caffeine, oriental raisin fruit extract, green tea extract, lonicera flower extract, peony extract, sophora root extract, scutellaria root, nutgall extract, peach leaf extract, ginko leaf extract and hyaluronic acid and further comprising one or more selected from a group consisting of acetyl hexapeptide-8, copper tripeptide-1, p-anisic acid and caprylyl glycol;
an eye cream applied after use of the lifting pack, comprising purified water, a glyceryl acrylate/acrylic acid copolymer and glycerin and further comprising one or more selected from a group consisting of propylene glycol, butylene glycol, betaine, PEG-8 dimethicone, Polysorbate 20, PPG-26-Buteth-26, cyclopentasiloxane, cyclotetrasiloxane, dimethiconol and potassium glycyrrhizate;
a whitening essence applied after use of the eye cream, comprising purified water, dipropylene glycol, squalane, glycerin and arbutin and further comprising one or more selected from a group consisting of phenyl trimethicone, a PEG-10 dimethicone/vinyl dimethicone crosspolymer, hydrogenated lecithin, cyclopentasiloxane, 1,2-hexanediol, polyglyceryl-3 methylglucose distearate, sodium hydrogenated tallow glutamate, a carbomer, ethoxydiglycol, xanthan gum and a trimethylpentanediol/adipic acid copolymer;
a nourishing cream applied after use of the whitening essence, comprising purified water, EGF, a peptide, dipropylene glycol, cetyl ethylhexanoate, a PEG-10 dimethicone/vinyl dimethicone crosspolymer and squalane and further comprising one or more selected from a group consisting of glyceryl stearate, dimethicone, 1,2-hexanediol, stearyl alcohol, meadowfoam seed oil, Polysorbate 60, ethoxydiglycol, a trimethylpentanediol/adipic acid copolymer, hydrogenated lecithin, sodium hydrogenated tallow glutamate, a carbomer and cetyl alcohol; and
a blemish balm or beauty balm cream applied after use of the nourishing cream, comprising purified water, caprylic/capric triglyceride, titanium dioxide, propylene glycol and squalane and further comprising one or more selected from a group consisting of cetyl PEG/PPG-10/1-dimethicone, arbutin, butylene glycol dicaprylate/dicaprate, sorbitan sesquioleate, magnesium sulfate, calcium stearate and microcrystalline wax.

8. The system according to claim 7, wherein the eye cream further comprises one or more selected from a group consisting of oriental raisin fruit extract, sodium hyaluronate, yeast β-glucan, acetyl hexapeptide-8, copper tripeptide-1, palmitoyl pentapeptide-4, PEG-40 hydrogenated castor oil, human oligopeptide-1, tripeptide-29, triethanolamine, adenosine, phenoxyethanol, 1,2-hexanediol benzophenone-5, diazolinyl urea and a fragrance.

9. The system according to claim 7, wherein the whitening essence further comprises one or more selected from a group consisting of tocopheryl acetate, oriental raisin fruit extract, sodium hyaluronate, sodium polyacrylate, yeast β-glycan, acetyl hexapeptide-8, copper tripeptide-1, palmitoyl pentapeptide-4, diipeptide-2, human oligopeptide-1, phenoxyethanol, triethanolamine, tetrasodium EDTA, a fragrance and a pigment.

10. The system according to claim 7, wherein the nourishing cream further comprises one or more selected from a group consisting of tocopheryl acetate, xanthan gum, sodium hyaluronate, triethanolamine, palmitoyl pentapeptide-4, acetyl hexapeptide-8, copper tripeptide-1, benzophenone-5, dipotassium glycyrrhizate, sodium polyacrylate, yeast β-glucan, C13-14 isoparaffin, laureth-7, polyacrylamide, human oligopeptide-1, adenosine, tetrasodium EDTA and a fragrance.

11. The system according to claim 7, wherein the blemish balm or beauty balm cream further comprises one or more selected from a group consisting of white gourd fruit extract, portulaca extract, aloe vera leaf juice, oriental raisin fruit extract, nylon-12, disteardimonium hectorite, triethoxycaprylylsilane, beeswax, poly(methyl methacrylate), tocopheryl acetate, cellulose gum, dimethicone, copper tripeptide-1, acetyl hexapeptide-8, palmitoyl pentapeptide-4, DMDM hydantoin, phenoxyethanol, red iron oxide, yellow iron oxide, black iron oxide, ultramarine, sodium lauroyl glutamate, aluminum hydroxide and a fragrance.

12. The system according to claim 7, which further comprise a formulation comprising Na₂O, SiO₂ and liquid sodium silicate which is mixed with the lifting pack before application to the skin.
